(19) United States
(12) Patent Application Publication
(10) Pub. No.: US 2023/0175109 A1
(43) Pub. Date: Jun. 8, 2023

(54) METHOD FOR SHAPING A SHAPE MEMORY WORKPIECE AND SHAPING TOOL FOR SHAPING A SHAPE MEMORY WORKPIECE

(71) Applicant: Admedes GmbH, Pforzheim (DE)

(72) Inventors: Stefan Zende, Karlsruhe (DE); Alexander Hegel, Karlsruhe (DE)

(21) Appl. No.: 18/062,866

(22) Filed: Dec. 7, 2022

(30) Foreign Application Priority Data
Dec. 8, 2021 (DE) 10 2021 006 050.4

(51) Int. Cl. C22F 1/00; C22F 1/10; F03G 7/06

(52) U.S. Cl. CPC C22F 1/006; C22F 1/10; F03G 7/06143; A61F 2240/001; B23K 2103/14

(57) ABSTRACT

A method for shaping a shape memory workpiece includes:
providing a shape memory workpiece having a first diameter and a predetermined shaping temperature;
arranging the shape memory workpiece on a shaping tool;
heating the shape memory workpiece to the shaping temperature;
first expansion of the shape memory workpiece to a second diameter that is larger than the first diameter;
first changing of the temperature of the shape memory workpiece to an intermediate temperature below or above the shaping temperature;
bringing the shape memory workpiece to the shaping temperature again;
second expansion of the shape memory workpiece to a third diameter that is larger than the second diameter;
ejecting the shape memory workpiece from the shaping tool; and
final cooling of the shape memory workpiece to a cooling temperature below the intermediate temperature.

A shaping tool is also provided.

### CROSS-REFERENCE TO RELATED APPLICATIONS

The present application claims priority to and the benefit of German Application No. 102021006050.4, filed Dec. 8, 2021. The disclosure of which is incorporated herein in its entirety.

### FIELD OF THE INVENTION

The present invention relates to a method for shaping a shape memory workpiece and a shaping tool for shaping a shape memory workpiece.

### BACKGROUND OF THE INVENTION

Shape memory alloys and shape memory workpieces produced therewith are known in various application scenarios, such as in medical technology. Depending on the environmental conditions, shape memory workpieces have different states and are characterized by the fact that even small changes in the environmental conditions can result in significant changes in the shape or geometry of the shape memory workpiece by the shape memory workpiece “remembering” a specific shape or a specific state. The characteristic properties of a shape memory workpiece can basically be divided into temperature-related shape memory properties and stress-related shape memory properties, with the stress-related shape memory properties also being described by term superelasticity or superelastic properties. The temperature-related shape memory properties as well as the superelastic properties and the associated states of a shape memory workpiece, such as in particular geometry and temperature and/or stress at which the geometry is assumed, are impressed on the shape memory workpiece in the course of its production.

Various methods are known for shaping shape memory workpieces, such as stents and heart valve frames, and the associated impressing of the temperature-related shape memory properties and/or the superelastic properties. The prior art proposes a method in which a shape memory workpiece is gradually expanded in a plurality of steps, with the expansion itself taking place at a temperature around room temperature and the shape memory workpiece being heated between the individual steps of expansion. In contrast, document EP 2 756 109 B1 as further prior art proposes a method for shaping a shape memory workpiece in which a shape memory workpiece is first heated to a shaping temperature in order to then expand the shape memory workpiece in a single step, in which the original diameter of the shape memory workpiece is increased by at least twice, up to six times. Subsequently, the shape memory workpiece is cooled down together with the shaping tool on which it is arranged for expansion in order thus to impress a desired shape memory behavior on the shape memory workpiece. Document EP 2 756 109 B1 compares the two methods outlined above in FIGS. 1 and 2 of document EP 2 756 109 B1.

However, various problems arise during the shaping of shape memory workpieces. Damage to the shape memory workpiece can occur during deformation, which prevents or complicates reliable impressing of the shape memory properties. For example, damage can be caused by locally excessive elongations on the shape memory workpiece. In addition, precise process control with regard to the reshaping or expansion of a shape memory workpiece and the environmental conditions applied in the process is very complex from an energetic point of view.

### SUMMARY OF THE INVENTION

It is therefore the object of the present invention to provide a method for shaping a shape memory workpiece that is improved in particular compared to document EP 2 756 109 B1. In addition, it is the object of the present invention to provide a shaping tool for improved shaping of a shape memory workpiece.

The object described above is solved by the subject matters of the independent claims; preferred embodiments are subject of the dependent claims.

One aspect of the invention relates to a method for shaping a shape memory workpiece, comprising:
providing a shape memory workpiece having a first diameter and a predetermined shaping temperature;
arranging the shape memory workpiece on a shaping tool;
heating the shape memory workpiece to the shaping temperature;
first expansion of the shape memory workpiece to a second diameter that is larger than the first diameter;
first changing of the temperature of the shape memory workpiece to an intermediate temperature below or above the shaping temperature;
bringing the shape memory workpiece to the shaping temperature again;
second expansion of the shape memory workpiece to a third diameter that is larger than the second diameter;
ejecting the shape memory workpiece from the shaping tool; and
final cooling of the shape memory workpiece to a cooling temperature below the intermediate temperature.

In other words, the above aspect relates to a method for shaping a shape memory workpiece, wherein in particular the shape memory workpiece is first heated to the shape memory temperature of the shape memory workpiece, and after a first expansion the shape memory workpiece is cooled or heated relative to the shape memory temperature. If the shape memory workpiece is cooled to an intermediate temperature after the first expansion, the shape memory workpiece assumes an intermediate temperature that is lower than the shape memory temperature to which the shape memory workpiece was originally heated and, in particular, is different from the shape memory temperature to which the shape memory workpiece was originally heated. If the shape memory workpiece is heated after the first expansion, the shape memory workpiece assumes an intermediate temperature that is higher than the shape memory temperature to which the shape memory workpiece was originally heated and, in particular, is different from the shape memory temperature to which the shape memory workpiece was originally heated. After a predetermined holding period of the shape memory workpiece at the intermediate temperature different from the shape memory temperature, the shape memory workpiece is brought back to the shape memory temperature, i.e., by heating or cooling, wherein exactly the shape memory temperature that corresponds to the shape memory temperature to which the shape memory workpiece was originally brought is substantially and preferably reached again. Subsequently, the shape memory workpiece can be expanded once more.

Advantageously, by several steps of expansion of the shape memory workpiece being carried out, the method according to the invention for shaping a shape memory workpiece enables the expansion of the shape memory workpiece starting from an initial or first diameter to a target diameter, here a third diameter, to be particularly gentle on the shape memory workpiece. Consequently, even with a large diameter ratio between the target diameter reached before the final cooling and the initial or first diameter, damage to the shape memory workpiece can be reduced or prevented. At the same time, by using several steps for expanding, the risk of springback of the geometry of the shape memory workpiece, which occurs particularly in the case of individual steps of expansion with a large diameter ratio, is advantageously prevented. As a result, this enables temperature-related shape memory properties or superelastic properties to be impressed on the shape memory workpiece in a very precise manner, in particular with regard to a geometry or a diameter that the shape memory workpiece is configured to assume at a specific ambient temperature, in terms of shape memory, by being heated to the so-called austenite finish temperature above which the structure of the shape memory workpiece is completely austenitic in the stress-free state. As a result, the present method is particularly suitable as a method for shaping shape memory workpieces that are subject to high demands on the accuracy of their deformation, their geometry, and their material and product properties within the shape memory, such as in the medical field e.g., for stents, heart valves, heart valve frames or similar parts. The suitability of the present method is not limited to this though.

Furthermore, the explicit change in the temperature of the shape memory workpiece to the intermediate temperature, which in preferred embodiments is below 500° C., preferably in the range from about 250° C. to about 500° C., or above 525° C., preferably in the range from about 525° C. to about 600° C., promotes the formation of precipitates in the structure of the shape memory workpiece, which in turn promotes the defined impressing of superelastic properties or shape memory properties. First changing the temperature of the shape memory workpiece may, in exemplary embodiments, comprise cooling the shape memory workpiece to an intermediate temperature below the shaping temperature. In other exemplary embodiments, first changing the temperature of the shape memory workpiece may comprise further heating the shape memory workpiece to an intermediate temperature above the shaping temperature. Consequently, bringing the shape memory workpiece to the shaping temperature may comprise reheating the shape memory workpiece to the shaping temperature in the event of cooling to the intermediate temperature, and cooling the shape memory workpiece to the shaping temperature in the event of further heating to the intermediate temperature.

In addition, the present method advantageously makes it possible to provide a particularly efficient shaping for a shape memory workpiece, in particular in terms of energy and time. By ejecting the shape memory workpiece from the shaping tool, such as into a cooling device, final cooling can be directed substantially to the shape memory workpiece while the shaping tool is cooled only little or not at all compared to the shape memory workpiece. Since the shaping tool is advantageously not ejected as well, the shaping tool does not have to be reassembled or completely reheated during a further run to shape another shape memory workpiece, since it already has a higher temperature than a newly provided shape memory workpiece. This advantageously enables a particularly time-, energy- and thus also cost-efficient method for shaping a shape memory workpiece.

By the shape memory workpiece being ejected from the shaping tool, the present method also enables the shape memory workpiece to be subjected to a particularly precisely defined cooling rate, since the thermally inert mass of the shaping tool does not adhere to the shape memory workpiece.

The present method thus enables shaping a shape memory workpiece in a particularly efficient manner such that shape memory properties with regard to diameter, temperature, and material and product properties of the shape memory workpiece can be set in a targeted manner, thereby providing an improved method for shaping a shape memory workpiece.

In exemplary embodiments of the method for shaping a shape memory workpiece, the step of ejecting the shape memory workpiece from the shaping tool can comprise, in particular, ejecting the shape memory workpiece from the shaping tool into a cooling device. The cooling device can be e.g., a basin or an area that is provided with a cooling medium (in particular a cooling liquid). For example, the cooling device can comprise or be a water basin.

In further exemplary embodiments of the present method, after the step of the second expansion, the method can include a step of a second changing of the temperature of the shape memory workpiece to an intermediate temperature below or above the shaping temperature, and subsequently, for example, a further step of again bringing the shape memory workpiece back to the shaping temperature, and furthermore, for example, in turn subsequently, a step of a third expansion of the shape memory workpiece to a fourth diameter that is larger than the third diameter.

Compared to the method in which the shape memory workpiece is ejected after the second expansion and is finally cooled, this makes it possible to expand the shape memory workpiece further and impress an even larger diameter for the shape memory workpiece, while in particular the risk of damage to the shape memory workpiece during deformation or during expansion is reduced.

In further exemplary embodiments of the method, the method can include any number of further steps of changing the temperature of the shape memory workpiece to an intermediate temperature below or above the shaping temperature, any (i.e. any number of) further steps of bringing the shape memory workpiece to the shaping temperature, and/or any (i.e. any number of) further steps of expansion to a diameter greater than in the previous step of expanding until a desired target diameter is substantially reached. This target diameter preferably represents about the diameter that the shape memory workpiece is intended to achieve in the course of a shape memory deformation. For example, the diameter that a stent is configured to assume when heated to about body temperature. Preferably, the target diameter represents a diameter which, to compensate for springback of the shape memory workpiece during final cooling, is slightly larger than the diameter that the shape memory workpiece is configured to assume when it is inserted into a body, for example. Since the specific applications of shape memory workpieces vary, for example with regard to the exact diameter of a vessel (e.g., a vein) for treatment with a stent, any desired diameter or any desired shape the shape memory workpiece is configured to assume at a specific temperature can be impressed on the shape memory workpiece as shape memory or as a superelastic property using a repeatable sequence of steps as described above. Furthermore, the repeatable sequence of steps described above enables defined product properties, such as the force exerted by the shape memory workpiece, in particular the force in the radial direction in the example of stents, at a specific deformation, and the proportion of plastic elongation at a specific deformation to be impressed on the shape memory workpiece.

In addition, the ejection of the shape memory workpiece after it has been expanded to a target diameter allows, in particular, that subsantially only the shape memory workpiece itself is cooled, so that the shape memory workpiece can be subjected to a particularly precisely defined cooling rate, and moreover an advantageously efficient (especially time- and cost-efficient) procedure is provided. A defined cooling rate also enables precisely defined shape memory properties or superelastic properties to be impressed on the shape memory workpiece.

In exemplary embodiments of the method for shaping a shape memory workpiece, the expansion can in particular comprise a radial expansion of the shape memory workpiece or a radial enlargement of a diameter of the shape memory workpiece, preferably a radially substantially uniform expansion or enlargement of a diameter of the shape memory workpiece. Furthermore, the expansion can comprise expansion by means of the shaping tool, for example.

In further exemplary embodiments, the final cooling of the shape memory workpiece can comprise quenching the shape memory workpiece. During the final cooling, the shape memory workpiece can preferably assume a diameter that substantially corresponds to the diameter that the shape memory workpiece assumes when the shape memory workpiece is introduced into the human body e.g., by means of a catheter and is heated over the austenite finish temperature.

Furthermore, in exemplary embodiments, the step or steps of heating the shape memory workpiece to the shaping temperature can comprise heating the shape memory workpiece and heating the shaping tool to the respective shaping temperature. For example, both the shape memory workpiece and the shaping tool can be heated together, for example in a salt bath. Alternatively, or additionally, one of the shape memory workpiece and the shaping tool can be heated such that the other of the shape memory workpiece and the shaping tool is directly or indirectly heated.

In the further course, various terms are used repeatedly, the understanding of which is to be made easier by the following exemplary descriptions.

Shape memory workpiece: The term shape memory workpiece refers in particular to a part or component that has or consists of a shape memory material, so that the shape memory workpiece has shape memory properties and/or superelastic properties at least in parts. Exemplary shape memory workpieces can be in particular stents, heart valves and other parts. The shape memory workpieces described for the aspects and embodiments described here can have or consist of any shape memory materials.

Superelastic properties describe a reversible change in shape caused by an external force. Superelastic deformation due to superelastic properties can exceed the elasticity of ordinary metals by a multiple. The cause of this behavior is a phase transformation within the shape memory material. Starting from an austenitic structure, the structure changes to a martensitic structure under stress. Due to the transformation, the shape memory workpiece can be present with different elongations at a predetermined stress, with the elongation of the austenitic structure being less than that of the martensitic structure at the same stress. When the stress is relieved, the martensite transforms back into austenite. No temperature changes are required for this. In contrast, there are temperature-related shape memory properties. Starting from a shape memory material that is present as a martensitic structure, a phase transformation into an austenitic structure can be completed by heating. This transformation is reversible. As of a so-called austenite finish temperature, the structure is as completely austenitic structure. In the case of stents and heart valves, for example, this austenite finish temperature is preferably reached and exceeded when they are introduced into the human body. Desired superelastic properties and shape memory properties can be impressed by thermally controlled shaping processes.

Exemplary shape memory materials can be nickel-titanium alloys, in particular nitinol. Alternatively, the shape memory material may be a ternary nickel-titanium alloy NiTiX, where X is, for example, copper, iron, niobium or chromium, or a polymer, or any other shape memory alloy.

Shaping temperature: The shaping temperature describes in particular a temperature range in which a shape memory workpiece, if it is exposed to a temperature of this range in a certain imposed shape or geometry for a predetermined time, retains this imposed shape or geometry even if the constraint condition is relieved after the predetermined time. The shaping temperature exemplary for the present method when the shape memory workpiece comprises or consists of nitinol is in a range from about 425° C. to about 550° C. in preferred embodiments. The shaping temperature to which the shape memory workpiece is brought influences the size and composition of the precipitates that can form in the structure of the shape memory workpiece, which can include in particular NiTi₂ and/or Ni₄Tis.

In exemplary embodiments, the lower shaping temperature range can be linked with heating to an intermediate temperature above the shaping temperature in terms of process technology. In alternative embodiments, the lower shaping temperature range can also be linked with cooling to an intermediate temperature below the shaping temperature in terms of process technology.

Furthermore, in exemplary embodiments, the upper shaping temperature range can be linked with cooling to an intermediate temperature below the shaping temperature in terms of process technology. In alternative embodiments, the upper shaping temperature range can also be linked with further heating to an intermediate temperature above the shaping temperature in terms of process technology.

Axial direction: The axial direction is used here in particular mainly to describe the shaping tool that extends substantially in an axial direction with a part of its components. Here, the axial direction can in particular be a direction that corresponds to a substantial extension of a traversing tube and/or a guide element of the shaping tool. The axial direction can also be a direction along which the traversing tube can be traversed and/or a direction along which adjacent disks of the shaping tool are spaced apart from one another. In addition, the axial direction can in particular be a direction that substantially corresponds to an extension and/or substantially to an axis of a cylindrical or about cylindrical shape memory workpiece.

Radial direction: The radial direction is used here in particular mainly in connection with the diameter of the shape memory workpiece and describes the substantial deformation of the shape memory workpiece during the one or more steps of expansion, for example by a shaping tool. The radial direction can preferably be substantially perpendicular to the axial direction. In other words, the radial direction can substantially correspond to a radial direction of a cylindrical or about cylindrical shape memory workpiece.

A circumferential direction can preferably be substantially perpendicular to the axial direction and/or substantially perpendicular to the radial direction.

The axial direction, the radial direction, and the circumferential direction can form a coordinate system, in particular a right-handed system.

If a direction or an angle is provided with the addition “substantially” or “about”, this addition means or is to be understood in particular as a deviation from the respective direction or from the respective angle in the range of 0° to 5°.

If a spatial dimension, a spatial relationship or any other relationship is provided with the addition “substantially” or “about”, this addition means or is to be understood in particular as a deviation from the respective dimension or the respective ratio in the range of 0% to 10%.

If a temperature is provided with the addition “substantially” or “about”, this addition means or is to be understood in particular as a deviation from the respective temperature in the range of 0% to 5% with respect to its equivalent in Kelvin. The term “room temperature” or “about room temperature” is to be understood in particular as a temperature in the range from about 15° C. to about 25° C. The term “about body temperature” means a temperature in the range of about 33° C. to about 42° C.

ln preferred embodiments of the method for shaping a shape memory workpiece, the first changing of the temperature of the shape memory workpiece to an intermediate temperature can comprise a first cooling of the shape memory workpiece to the intermediate temperature below the shaping temperature, or a first further heating of the shape memory workpiece to the intermediate temperature above the shaping temperature, and the first changing of the temperature of the shape memory workpiece can preferably comprise changing the temperature of the shape memory workpiece by at least about 25° C., preferably by at least about 40° C., more preferably by at least about 50° C.

Advantageously, changing the temperature of the shape memory workpiece to the intermediate temperature, by cooling or further heating the shape memory workpiece, promotes the formation of precipitates in the structure of the shape memory workpiece, which in turn promotes the defined impressing of superelastic properties or shape memory properties. Changing the temperature of the shape memory workpiece by at least about 25° C., preferably by at least about 40° C., more preferably by at least about 50° C. further enhances the aforementioned effect of forming precipitates.

Thus, according to an exemplary embodiment, the shape memory workpiece can be heated to a temperature of about 500° C., for example, in the step of heating the shape memory workpiece to the shaping temperature, and in the step of first changing the temperature of the shape memory workpiece to an intermediate temperature below or above the shaping temperature, for example to a temperature of about 465° C.

ln preferred embodiments of the method for shaping a shape memory workpiece, the second diameter can be about 1.5 times to about 1.9 times the first diameter, preferably about 1.85 times the first diameter. Additionally, or alternatively, the third diameter can be about 1.5 times to about 1.9 times the second diameter, preferably about 1.85 times the second diameter.

Advantageously, the aforementioned diameter ratios that are achieved in particular during the expansion steps make it possible that, starting from an initial or first diameter of a shape memory workpiece and to achieve a large target diameter of the shape memory workpiece, only a few steps or a very limited number of expansion steps are required on the one hand, and damage to the shape memory workpiece can be reduced or avoided on the other hand. Consequently, the aforementioned diameter ratios make it possible for the shape memory properties of the diameter or shape and temperature of the shape memory workpiece to be impressed on the shape memory workpiece with pinpoint accuracy. The aforementioned diameter ratios are also particularly suitable for expansion steps that go beyond the first expansion and the second expansion, i.e., for example, for a third expansion, fourth expansion, etc. In other words, the preferred diameter ratios mentioned above allow an advantageous compromise between process reliability for defining the desired product properties of the shape memory workpiece and process economy by requiring only a few individual steps to expand the shape memory workpiece.

In exemplary embodiments, the aforementioned diameter ratios can also be above or below average in order to set a target diameter as precise as possible for the shape memory workpiece. If the diameter ratio falls below 1.9, critical locally excessive elongations on the shape memory workpiece can advantageously be reduced or avoided when expanding the shape memory workpiece, and further damage to the shape memory workpiece can thereby be restricted or avoided.

ln preferred embodiments of the method for shaping a shape memory workpiece, the first expansion and the second expansion can comprise an expansion of the shape memory workpiece in the radial direction, in particular while maintaining the axial extension of the shape memory workpiece.

Advantageously, maintaining the axial extension of the shape memory workpiece during the expansion allows the change in shape of the shape memory workpiece due to the expansion in the radial direction substantially or only to refer to the diameter of the shape memory workpiece. In other words, it is advantageously made possible that, during the expansion, there is little or no compression of the shape memory workpiece, which has a negative impact on the desired geometry with regard to the shape memory. Maintaining the substantially axial extension of the shape memory workpiece during expansion also makes it possible to avoid costly and complicated rework on the shape memory workpiece.

Maintaining the axial extension of the shape memory workpiece is to be understood in particular as avoiding uncontrolled axial deformation of the shape memory workpiece during expansion in the radial direction. In the present method, the axial deformation is preferably limited such that after the expansion, the shape memory workpiece expands at least about 0.70 times, preferably about 0.80 times, more preferably about 0.90 times, even more preferably at least that about 0.95 times, and more preferably at least about 0.98 times of its axial extension prior to expansion. Depending on the specific shape memory workpiece and the degree of reshaping applied to the specific shape memory workpiece, however, deviating limits of axial deformation can also be permitted.

In exemplary embodiments, each step of expanding can comprise expanding the shape memory workpiece while maintaining the axial extension of the shape memory workpiece.

In further exemplary embodiments, the expansion of the shape memory workpiece can in particular also comprise expanding the shape memory workpiece in the radial direction while maintaining the axial arrangement of the shape memory workpiece on the shaping tool.

In particular, this advantageously enables not only the axial extension or the axial geometry of the shape memory workpiece to be adjusted in a defined manner during the expansion, but also the arrangement of the shape memory workpiece on the shaping tool to be retained. This further advantageously enables the shape memory workpiece to be arranged on a predetermined area of the shaping tool from which it is not displaced as a result of the expansion. In this way, the steps of cooling and/or heating can be carried out in a specifically restricted area.

Furthermore, this makes it possible in particular to provide an energetically particularly efficient method for shaping one or more shape memory workpieces, wherein the thermally relevant and thus energetically intensive steps of cooling and/or heating can be concentrated on the area in which the shape memory workpiece is arranged on the shaping tool, independent of the exact number of heating, expanding and/or cooling steps or cycles performed.

In exemplary embodiments, maintaining the axial extension of the shape memory workpiece and/or maintaining the axial arrangement of the shape memory workpiece on the shaping tool can be ensured by positive and/or frictional holding, such as by gripping or holding hooks, stops, grippers or the like.

In preferred embodiments of the method for shaping a shape memory workpiece, the shape memory workpiece can be held in a frictional manner during at least one of the first and second expansions, in particular it can be held in a frictional manner on the shaping tool.

In addition, in exemplary embodiments, the shape memory workpiece can be frictionally held or localized in or during each step of expanding.

Advantageously, the frictional holding or localization of the shape memory workpiece during expansion allows the shape memory workpiece to be held on the shaping tool in a particularly simple manner.

In further exemplary embodiments, the frictional holding or localization can in particular include frictional holding or localization while maintaining the axial extension of the shape memory workpiece, particularly preferably while maintaining the axial position of the shape memory workpiece on the shaping tool.

The frictional holding or localization of the shape memory workpiece makes it possible to ensure that the shape memory workpiece is held in its axial extension and/or in its axial arrangement on the shaping tool in a particularly simple manner, for example using a predetermined material pair.

In preferred embodiments, the frictional holding or localization can be based on static friction, in particular on static friction between the shape memory workpiece and the shaping tool.

In particular, the static friction between the shape memory workpiece and the shaping tool advantageously allows the shape memory workpiece to be expanded with the largest possible expansion angle, for example compared to sliding friction. This also advantageously allows the method for shaping the shape memory workpiece to be carried out in a particularly time-efficient manner, as well as a compact structure for the shaping tool, which in turn results in increased energy efficiency.

In particularly preferred embodiments, the frictional connection can be configured between a shape memory workpiece comprising or consisting of nitinol and a plurality of expansion wires or expansion elements comprising or consisting of nitinol. The material pair nitinol-nitinol for configuring a frictional holding of the shape memory workpiece advantageously prevents contamination of the shape memory workpiece to be formed even at high temperatures, further prevents contamination of a possible salt bath for heating the shape memory workpiece, and also enables temperature-insensitive, high static friction. Furthermore, a shaping tool comprising nitinol provides advantageous elasticity over a wide temperature range. A preferred expansion angle, measured in relation to a substantially axial direction, can be in the range from about 7° to about 20°, preferably in the range from about 10° to about 16°, for a nitinol-nitinol material pair.

In alternative embodiments, material pairs differing therefrom can be used to configure a frictional connection.

ln preferred embodiments of the method for shaping a shape memory workpiece, the shape memory workpiece can:
have a nickel-titanium alloy; and/or
have a round shape; and/or
have a stent pattern.

With a shape memory workpiece that comprising or consisting of a nickel-titanium alloy such as nitinol, the shape memory workpiece can particularly be suitable for medical applications.

Due to a preferably round shape of the shape memory workpiece, it can be expanded uniformly in a particularly simple manner in the radial direction, i.e., to a specific, discrete diameter. The shape or basic shape of the shape memory workpiece can in particular be substantially cylindrical or substantially ring-shaped, but is not limited to an exactly round shape, but can also be polygonal or oval, for example.

Due to a preferred stent pattern, the shape memory workpiece is particularly suitable for medical use or for use as a stent or implant. The stent pattern can be designed as a metallic mesh with a predetermined pattern. Furthermore, the stent pattern can be shaped in particular by punching out of a metal plate, as well as by removing material (e.g., by means of laser cutting) of a metal plate or a metal cylinder or tube, or the like.

In alternative embodiments, the shape memory workpiece can deviate from a round shape and/or a stent pattern or have a shape that deviates therefrom. For example, the shape memory workpiece can have any desired polygonal shape, for example similar to a polygonal tube. In addition, the shape memory workpiece can, in parts, have areas with a reduced or increased diameter, constrictions, indentations or bulges and conical areas.

Another aspect of the invention relates to a shaping tool for shaping a shape memory workpiece, having:
a guide element, and
a traversing tube, which is movably arranged on the guide element, wherein
   disks are arranged on the traversing tube at predetermined axial distances,
   expansion wires or expansion elements are stretched between the disks, and wherein
   the expansion wires or expansion elements stretched between the disks form diameters in order to arrange a shape memory workpiece circumferentially thereon.

Advantageously, the present shaping tool enables a shape memory workpiece to be held securely on its inner circumference by means of a plurality of expansion wires or elements. At the same time, the wire-like or element-like construction of the shaping tool, in order to arrange the shape memory workpiece thereon and also expand it further, enables the shaping tool to have a particularly low thermal capacity, so that the present shaping tool enables a particularly energy-efficient shaping of a shape memory workpiece.

In addition, the present shaping tool, by providing a plurality of expansion wires or elements to arrange a shape memory workpiece thereon, enables a corresponding plurality of holding points or mounting points to be provided between the shape memory workpiece and the shaping tool. This enables a particularly secure and defined holding during an exemplary expansion of the shape memory workpiece.

In exemplary embodiments of the shaping tool, the guide element can extend substantially in a longitudinal direction, in particular substantially in an axial direction. For example, the guide element can be designed as a guide rod, guide tube or the like. Furthermore, the traversing tube can e.g., substantially extend in an axial direction, or can substantially extend parallel to the guide element at least in parts.

In further exemplary embodiments, the expansion wires or elements can be arranged on or extend between the disks such that the shape memory workpiece, when it is arranged on the shaping tool, is only contacted by the expansion wires or elements. The shaping tool can have any number of expansion wires or elements, for example 3, 4, 5, 6, 7, 8, 9, 10, 11, 12, 13, 14, 15, 16, 17, 18, 19, 20, or any greater plurality of expansion wires or elements.

In exemplary embodiments, the expansion wires or elements can be arranged substantially along an outer circumference of the disks or run at least in parts along an outer circumference of the disks. In this way, the expansion wires or elements can e.g., be arranged on the disks such that the expansion wires or elements project outwards in the radial direction, at least in parts, in relation to the disks. In other words, the expansion wires or elements can e.g., be arranged on the disks in their longitudinal extent such that their wire diameter at least partially protrudes radially with respect to the diameter of the disks on which they are arranged or between which they are clamped.

The expansion wires or elements can preferably substantially form a lattice structure, in particular a lattice structure of elongated lattice elements spaced from one another in a circumferential direction.

Depending on the geometry of the disks between which the expansion wires or elements are stretched or between which the expansion wires or elements extend, the expansion wires or elements form e.g., a lattice structure in parts. Parts of the lattice structure can be formed from one or more cylindrical portions and one or more frustoconical portions. In particular, the one or more cylindrical portions can alternate with the one or more frustoconical portions.

In exemplary embodiments, the expansion wires or elements can in particular form a lattice structure that has a first substantially cylindrical portion having a first discrete diameter, an adjoining first substantially frustoconical portion forming a transition from the first discrete diameter to a second discrete diameter, and a second substantially cylindrical portion having the second discrete diameter, which adjoins the first substantially frustoconical portion and is larger than the first discrete diameter.

Further exemplary embodiments may form a lattice structure having one or more further substantially frustoconical portions each forming a transition between two diameters and one or more further substantially cylindrical portions forming a discrete diameter greater than a previous discrete diameter. In particular, further exemplary embodiments can form a lattice structure using the expansion wires or elements, which has a first substantially cylindrical portion having a first discrete diameter, an adjoining first substantially frustoconical portion forming a transition from the first discrete diameter to a second discrete diameter, a second substantially cylindrical portion having the second discrete diameter, which adjoins the first substantially frustoconical portion and is larger than the first discrete diameter, an adjoining second substantially frustoconical portion forming a transition from the second discrete diameter to a third discrete diameter, and a third substantially cylindrical portion having the third discrete diameter, which adjoins the second substantially frustoconical portion and is larger than the second discrete diameter.

The transition between two discrete diameters can particularly preferably be configured by a predetermined expansion angle in order to particularly ensure a frictional connection, preferably a frictional connection through static friction of a shape memory workpiece on the plurality of expansion wires or elements.

The first diameter formed by the first cylindrical portion having the first diameter may in particular be configured to hold the shape memory workpiece under a prestress thereon. The prestress can be so marginal that it only serves to ensure that the shape memory workpiece does not slip off by itself. In other words, the shape memory workpiece can be arranged on the first cylindrical section having the first diameter in particular by applying an elastic deformation, with an elastic restoring force of the shape memory workpiece holding the shape memory workpiece on the first cylindrical portion.

Furthermore, additionally or alternatively, the expansion wires or elements can in particular form a lattice structure having an oval or substantially polygonal cross section, such as a hexagonal cross section, in parts.

The exemplary and preferred embodiments described above thus advantageously make it possible to provide a shaping tool that enables the gradual expansion of a shape memory workpiece in a simple manner. Furthermore, a method for shaping a shape memory workpiece can be carried out in a particularly energy-efficient manner by means of the shaping tool described above, since the shaping tool has a particularly low thermal capacity due to its lattice-like structure.

In preferred embodiments of the shaping tool for shaping a shape memory workpiece, the shaping tool can have a receiving area for arranging thereon a shape memory workpiece having a first diameter, and
the traversing tube can be configured to be moved relative to the receiving area by means of an actuator, so that the shape memory workpiece can be passed by the disks in the receiving area.

In exemplary embodiments, the receiving area can be formed by expansion wires or elements that form a first receiving diameter in order to arrange thereon a shape memory workpiece having a first or initial diameter.

In other words, the receiving area can be configured in particular as an initial receiving area, which initially forms or has a first receiving diameter for arranging a shape memory workpiece having an initial or first diameter thereon, and, by the passing of the disks, is configured to form or have a diameter that is increased or decreased according to the passing disks.

Due to the passing of the disks, the expansion wires or elements can move in particular in the radial direction, according to the diameter or the geometry of the passing disks, whereby the plurality of expansion wires or elements distributed in the circumferential direction form a diameter corresponding to the passing disk. Since the shape memory workpiece is arranged circumferentially on the expansion wires or elements, the shape memory workpiece can thus be held in an adjustable or configurable manner independently of the process of deformation or expansion of the shape memory workpiece.

For a deformation or expansion of the shape memory workpiece, a relative movement between the shape memory workpiece and the expansion wires or elements can thus be prevented in an advantageous manner. As a result, the shape memory workpiece can be preferably held on the basis of static friction between the shape memory workpiece and the expansion wires or elements, which is independent or unaffected by the passing disks that are moved by the actuator.

In preferred embodiments of the shaping tool for shaping a shape memory workpiece, the disks can be shaped such that the shaping tool is configured to expand the shape memory workpiece by moving the traversing tube along the guide element.

In exemplary embodiments, the traversing tube can be traversable parallel to the guide element or parallel to the extension of the guide element, in particular it can be movable along an axial direction.

In exemplary embodiments, the disks can be shaped such that when passing the receiving area in the axial direction, the disks expand the expansion wires or elements radially or press them radially outward. In other words, the disks arranged on the traversing tube could be shaped such that each successive disk opposite to the axial direction has at least the same diameter as the disk arranged in front of it.

In exemplary embodiments, at least two disks having a first disk diameter can be arranged in succession opposite to the axial direction, followed by at least two disks having a second disk diameter that is larger than the first disk diameter. In further exemplary embodiments, one or more disks may be arranged between the disks having the first and second diameters and may have a disk diameter that is between the first and second disk diameters. The arrangement of further disks in this sense is possible, for example.

Due to the passing of the disks having a predetermined diameter, and preferably at a predetermined speed, it is advantageously possible to apply a defined reshaping speed, and thus above all a defined force, to the shape memory workpiece during shaping or expanding.

In preferred embodiments of the shaping tool for shaping a shape memory workpiece, the disks can have sliding grooves along their circumference,
an expansion wire or element being slidably guided by the respective disk along or in each sliding groove, and
each expansion wire or element being preferably arranged in the respective sliding groove such that it projects radially outward with respect to the disk in the sliding groove of which the expansion wire or element is guided.

Advantageously, the sliding guidance of the expansion wires in sliding grooves of the disks allows only the disks to be moved in order to allow expansion of the shape memory workpiece, while at the same time a relative movement between the expansion wires and the shape memory workpiece is avoided, which in turn allows expansion with a large expansion angle and further allows axial shortening of the shaping tool, which in turn increases energy efficiency.

The sliding grooves can be configured e.g., as bores, in particular as bores in the axial direction through the disk(s).

In exemplary embodiments, the disks have the sliding grooves in particular along their outer circumference.

In further exemplary embodiments of the shaping tool, the expanding wires or elements can be radially secured or radially fixed in the sliding grooves. In other words, the expansion wires or elements can be guided in the sliding grooves such that they are secured against movement radially outward. The radial fixation or the radial securing of the expansion wires or elements can e.g., be an actuatable clamp, or be formed geometrically by the sliding groove itself. The radial securing of the expansion wires or elements advantageously allows a particularly precise guidance of the expansion wires or elements, so that a targeted, step-by-step expansion to defined diameters is ensured and, furthermore, a targeted expansion angle can be set. In addition, the radial securing of the expansion wires or elements allows to prevent a local superimposition of two or more expansion wires or elements, which in particular helps to prevent locally critical deformations or elongations on the shape memory workpiece during shaping or during expansion.

In further exemplary embodiments, the disks can have a concave shape or a radially inward recess along their outer circumference and between two adjacent sliding grooves. The concave shape or the radially inward recess between two adjacent sliding grooves further ensures that the movable disks and the shape memory workpiece do not contact, whereby an axial extension of the shape memory workpiece and/or an axial arrangement of the shape memory workpiece on the shaping tool during expansion can be maintained.

In addition, the concave shape or the radial recesses between two adjacent sliding grooves allow the thermal capacity of the shaping tool to be further advantageously reduced.

In preferred embodiments of the shaping tool for shaping a shape memory workpiece, the shaping tool can further:
have a heating device, preferably a heatable salt bath; and/or
have a cooling device, preferably a water bath.

With the heating device, a device for heating, in particular for repeated or renewed heating or for further heating of the shape memory workpiece is advantageously provided. The heating device can e.g., comprise a bath having a heating medium, such as a salt bath. Alternatively, or additionally, the heating device can comprise other high-temperature liquids, as well as an inductive, convective, electrical resistance-based, radiation-based heating means, such as lasers or the like, or a combination thereof.

A device for cooling is advantageously provided by the cooling device. The cooling device can comprise one or more fans, a fluidic cooling medium and/or a water bath, for example. Alternatively, or additionally, the cooling device can comprise a gas container with gas, in particular with inert gas such as nitrogen, argon, etc., which is stored under pressure, for example, and flows out toward the shape memory workpiece if required.

While one or more fans are particularly suitable for lowering a shape memory workpiece to an intermediate temperature below a shaping temperature, a water bath is particularly suitable for final cooling of a shape memory workpiece to a temperature still below the intermediate temperature and, for example, by quenching to about room temperature or an even lower temperature. A gas container, which includes inert gas, for example, can be particularly suitable for both cooling processes.

In alternative embodiments, the heating device and/or the cooling device can be provided as external devices or can be arranged on and/or in the vicinity of the shaping tool.

In further exemplary embodiments of the shaping tool, the shaping tool can have an ejection device configured to eject the shape memory workpiece from the shaping tool, for example into a cooling device. The ejection device can comprise a lever or the like, for example.

In preferred embodiments, the ejection device is formed integrally by the traversing tube, in particular by the traversing tube and the actuator, which can be configured to eject the shape memory workpiece by moving or traversing the traversing tube in the opposite direction for expansion, which advantageously means that no further ejection elements are necessary.

Another aspect of the invention relates to a method for shaping a shape memory workpiece, similar to the aforementioned method for shaping a shape memory workpiece. The method for shaping a shape memory workpiece of the further aspect comprises:
providing a shape memory workpiece having a first diameter and a predetermined shaping temperature;
arranging the shape memory workpiece on a shaping tool;
heating the shape memory workpiece to the shaping temperature;
expanding the shape memory workpiece to a second diameter that is larger than the first diameter;
ejecting the shape memory workpiece from the shaping tool; and
final cooling of the shape memory workpiece to a cooling temperature.

The method for shaping a shape memory workpiece of the further aspect includes, in particular in comparison to the aforementioned method for shaping a shape memory workpiece, only one step of heating to the shaping temperature, only one step of expanding the shape memory workpiece, and in particular no step of changing the temperature of the shape memory workpiece to an intermediate temperature, i.e. in particular no step of cooling or further heating to an intermediate temperature.

The method of the further aspect advantageously makes it possible to provide a particularly time- and energy-efficient method for shaping a shape memory workpiece, in particular since the shape memory workpiece is ejected from the shaping tool in order to cool the shape memory workpiece to the cooling temperature. Thus, excessive cooling of the shaping tool is prevented, so that in a subsequent method for shaping a shape memory workpiece an advantageously reduced energy consumption is made possible, in which the shaping tool is already heated compared to another shape memory workpiece to be shaped, so that in particular the shape memory workpiece can also be heated more quickly. Furthermore, the ejection of the shape memory workpiece from the shaping tool allows the shape memory workpiece to be subjected to a precisely defined cooling rate, since no thermally inert shaping tool adheres to the shape memory workpiece, which in turn improves the accuracy of the setting of the product properties of the shape memory workpiece in particular with regard to geometry, austenite finish temperature, as well as the forces occurring during deformation. Finally, the ejection of the shape memory workpiece also avoids having to perform a separate step of separating the shape memory workpiece from the shaping tool, which further improves the process economy and, moreover, enables the process to be automated.

The preferred, exemplary and alternative embodiments relating to the preceding method and the preceding shaping tool, together with their advantages, also relate equally to the method of the further aspect.

Similarly, the preferred, exemplary and alternative embodiments relating to the method of the first and further aspects, together with their advantages, relate equally to the shaping tool and vice versa.

### BRIEF DESCRIPTION OF THE FIGURES

Embodiments of the invention will be described in more detail below with reference to the accompanying figures. It goes without saying that the present invention is not limited to these embodiments and that individual features of the embodiments can be combined to form further embodiments within the scope of the appended claims.

The figures show:

FIG. 1 a flowchart for the method for shaping a shape memory workpiece;

FIG. 2 a sketch of a portion of a shaping tool;

FIG. 3 a sketch of a portion of a disk of a shaping tool;

FIG. 4a a sectional representation of a shaping tool in a first state;

FIG. 4b a sectional view of a shaping tool in a second state;

FIG. 4c a sectional representation of a shaping tool in a third state; and

FIG. 5 a sketched temperature-time profile according to an example of the present method.

### DETAILED DESCRIPTION OF EMBODIMENTS OF THE INVENTION

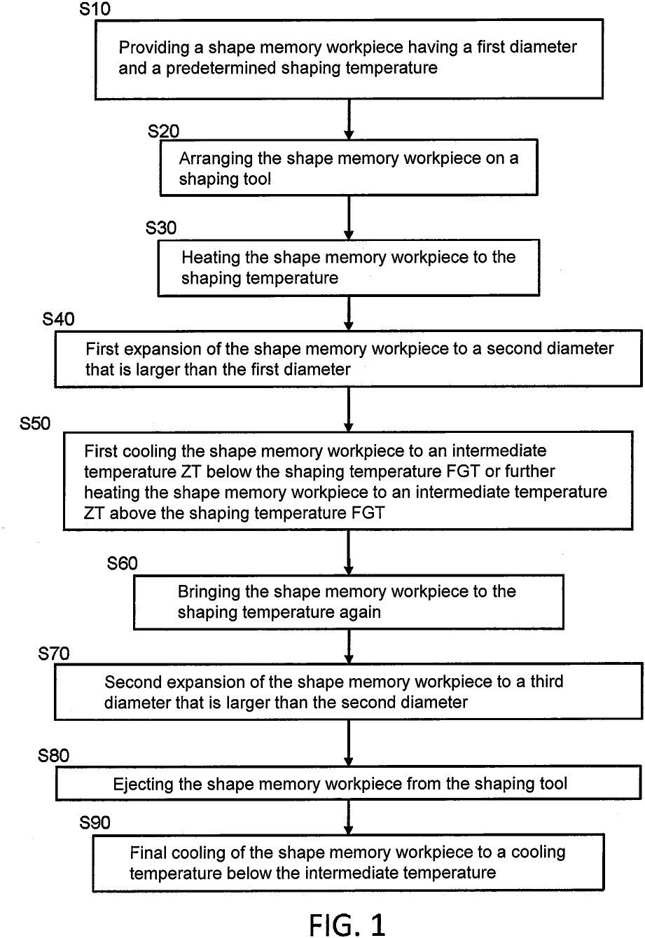

FIG. 1 shows a flowchart of an exemplary method, comprising the steps of:
S10: providing a shape memory workpiece 100 having a first diameter and a predetermined shaping temperature FGT;
S20: arranging the shape memory workpiece 100 on a shaping tool 10;
S30: heating the shape memory workpiece 100 to the shaping temperature FGT;
S40: first expansion of the shape memory workpiece 100 to a second diameter that is larger than the first diameter;
S50: first cooling of the shape memory workpiece 100 to an intermediate temperature ZT below the shaping temperature FGT or further heating of the shape memory workpiece 100 to an intermediate temperature ZT above the shaping temperature FGT;
S60: bringing the shape memory workpiece 100 to the shaping temperature FGT again;
S70: second expansion of the shape memory workpiece 100 to a third diameter that is larger than the second diameter;
S80: ejecting the shape memory workpiece 100 from the shaping tool 10; and
S90: final cooling of the shape memory workpiece 100 to a cooling temperature below the intermediate temperature ZT.

The method of FIG. 1 depicts an exemplary method for shaping a shape memory workpiece 100. Due to the multiple steps of expansion S40 and S70, wherein the shape memory workpiece 100 is expanded at a shaping temperature FGT of the shape memory workpiece 100, and the intermediate step of cooling or further heating to a temperature below or above the shaping temperature FGT, the overall deformation of the shape memory workpiece 100 for substantially obtaining a target shape or a target diameter is advantageously divided. By dividing the deformation into several steps of expansion, even large changes in diameter, starting from a first or initial diameter to a target diameter or a final diameter, can advantageously be implemented, with damage to the shape memory workpiece 100 advantageously being reduced or prevented.

At the same time, ejecting the shape memory workpiece 100 in step S80, after expanding to the target diameter or the third diameter in the sense of step S70, enables the final cooling in step S90 to be directed specifically at the shape memory workpiece 100, whereby an energetically time-consuming cooling and possibly reheating of the shaping tool 10 can be prevented. Furthermore, thermal stresses on the shaping tool 10 are advantageously reduced as a result.

In addition, the steps S30 and S60 of heating or bringing the shape memory workpiece 100 to the shaping temperature FGT can in particular comprise heating or bringing the shape memory workpiece 100 to the shaping temperature FGT and heating or bringing the shaping tool 10 to the shaping temperature FGT. Furthermore, in exemplary embodiments, either the shaping tool 10 or the shape memory workpiece 100 can be indirectly heated or cooled by heating or cooling the respective other one of the shaping tool 10 and the shape memory workpiece 100.

The method of shaping a shape memory workpiece 100 is not limited to the steps shown in FIG. 1. Instead, to achieve a specific target diameter or final diameter, one or more further steps of cooling or further heating to an intermediate temperature ZT below or above the shaping temperature FGT, bringing the shape memory workpiece 100 to the shaping temperature FGT again, and expanding the shape memory workpiece 100 to a diameter that is increased compared to the previous expanding step can be carried out, based on the respective steps S50, S60 and S70 according to FIG. 1. Preferably such that a target shape or a target diameter of the shape memory workpiece 100 is reached with the last step of expanding the shape memory workpiece 100, and so that steps S80 and S90 can follow next. The target diameter of the shape memory workpiece 100 can be a diameter that is larger than a predetermined diameter the shape memory workpiece 100 is configured to assume in use, for example after being inserted into a human body, in order to compensate for a possible springback of the shape memory workpiece 100 when it cools down after being ejected from the shaping tool 10.

In alternative embodiments, the method can be limited to steps S10, S20, S30, S40, S80 and S90, so that the shape memory workpiece 100 is expanded by means of a single step starting from a first or initial diameter to a target diameter or final diameter. Such a method provides a particularly fast and energy-efficient method for shaping a shape memory workpiece 100, which at the same time enables the product properties of the shape memory workpiece, such as in particular geometry and austenite finish temperature, to be set precisely by means of a precisely definable cooling rate.

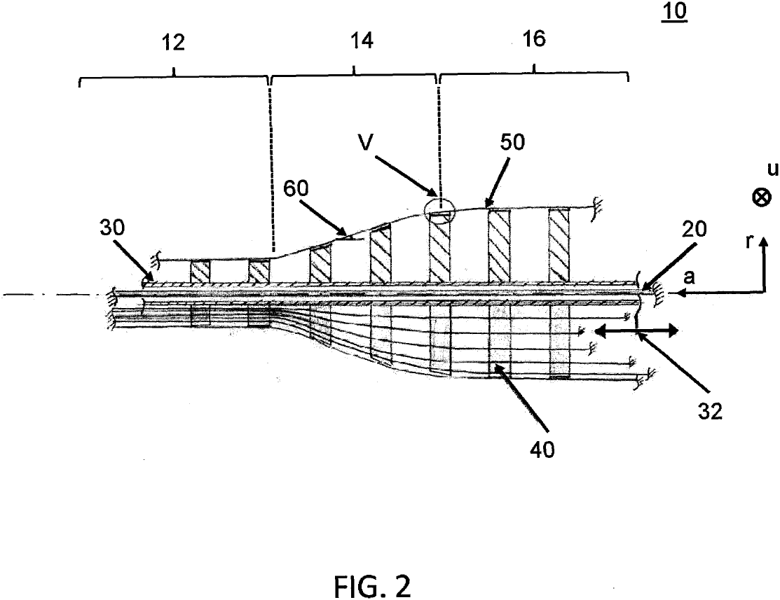

FIG. 2 shows a sketch of a portion of a shaping tool 10. Also shown in FIG. 2 is a coordinate system which in particular has the axial direction a, the radial direction r and the circumferential direction u. In the representation of FIG. 2, the axial direction a extends substantially from right to left, the radial direction r substantially extends radially outward from the axial direction a, shown exemplarily from bottom to top, and the circumferential direction u extends along a circumference of a preferably circular element of the shaping tool 10, shown exemplarily as pointing into the drawing plane.

The shaping tool 10, as shown in FIG. 2, in particular has a guide element 20 and a traversing tube 30, which is traversably arranged on the guide element 20. The traversing tube 30 can be supported at least partially in a sliding manner on the guide element 20. As shown in FIG. 2, the guide element 20 can extend substantially in the axial direction a and can be formed by a rod or hollow cylinder, for example.

The traversing tube 30 preferably extends at least in parts parallel to the guide element 20. In other words, the traversing tube 30 can extend at least in parts along the axial direction a. The traversing tube 30 can in particular be hollow, preferably hollow-cylindrical, and accommodate the guide element 20 at least in parts.

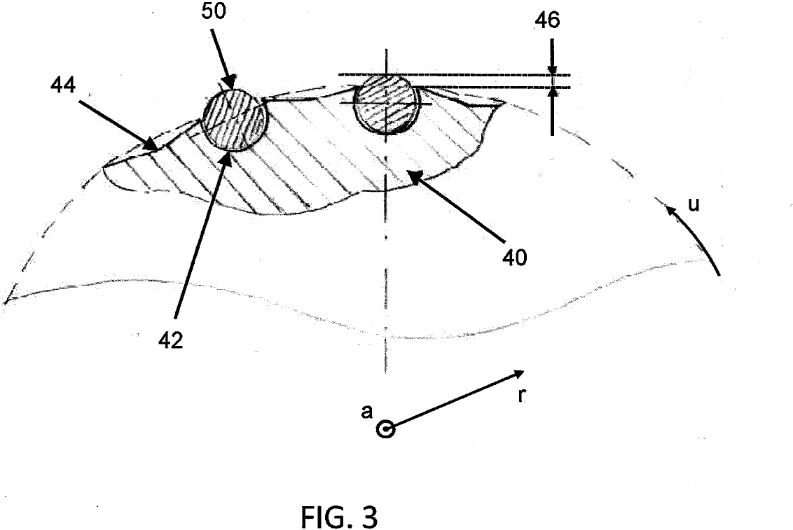

As further shown in FIG. 2, several disks 40 are arranged on the traversing tube 30, and expansion wires or (e.g., web-like, flexible) expansion elements 50 are arranged or attached such that they are stretched between the disks 40 or extend between the disks 40. As further illustrated in FIG. 3, the expansion wires or elements 50 preferably run along an outer circumference of the disks 40 or through sliding grooves 42 of the disks 40.

The disks 40 are preferably arranged along the traversing tube 30 at predetermined axial distances from one another, i.e., distances substantially along the axial direction a. The disks 40 can be arranged substantially equidistant from one another or have a varying distance from one another. The shaping tool 10 can have any number of disks 40 or any number of disks 40 can be arranged on the traversing tube 30.

The number of disks 40 arranged on the traversing tube 30 can correspond at least to the number of different diameters that a shape memory workpiece 100 to be shaped gradually assumes in the method for shaping a shape memory workpiece 100.

In addition, in a transition area 14 between two discrete diameter areas 12, 16, each of which is formed by a plurality of disks 40 with the same diameter, no disk 40 or any number of disks 40 can be arranged on the traversing tube 30 to support the transition area 14 and to ensure a defined expansion angle 60, also during the formation of a shape memory workpiece 100.

The expansion angle 60 occurs as an angle in the transition area 14 of the shaping tool 10, in particular as an angle following the expansion wire 50 in relation to the axial direction a. The expansion angle 60 is determined by the diameter of two consecutive disks 40 and the axial distance between the two consecutive disks 40 in question. The expansion angle 60 thus describes, in combination with a traversing speed of the traversing tube 30, the deformation per time or the change in diameter per time, and thus the force applied to the shape memory alloy 100.

The expansion angle 60 thus influences the process time and also very significantly the geometry of the shaping tool 10, since the larger the expansion angle 60, the shorter the shaping tool 10 can be realized, which in turn contributes to reducing the use of material for the shaping tool 10, which reduces its thermal capacity and increases the energy efficiency of the process. Furthermore, a shorter shaping tool 10 with a correspondingly comparatively low thermal capacity allows a heating device to be of compact design, and as a result in turn has a comparatively low power loss, which in turn finally increases the energy efficiency in the process and also the energy efficiency of the shaping tool 10. On the other hand, a large expansion angle 60 causes the shape memory workpiece 100 to slip off the shaping tool 10 comparatively more easily, since the static friction between the shape memory workpiece 100 and the expansion wires or elements 50 decreases during the expansion as the expansion angle 60 increases.

As outlined in FIG. 2 by means of the braces, substantially three different characteristic portions can be formed on the shaping tool 10. Here, a receiving area 12 can form in particular on an axial first portion of the shaping tool 10, on which disks 40 in cooperation with expansion wires or elements 50 arranged or tensioned thereon form a first discrete diameter that is suitable for shape memory workpieces 100 having a first or initial diameter to be arranged on it, in particular to be arranged under prestress on it. The first or initial diameter of the shape memory workpiece 100, and thus the first discrete diameter formed by the disks 40 with the expansion wires 50 arranged thereon in the receiving area 12, can be, for example for stents, in a range from about 3 mm to about 15 mm, preferably in a range from about 6 mm to about 12 mm. However, neither stents nor other exemplary shape memory workpieces, such as heart valves, are limited to the aforementioned diameters. Adjoining the receiving area 12 and following in the opposite direction of the axial direction a, a transition area 14 can e.g., extend on the shaping tool 10, which forms a transition between the first and a second discrete diameter. A disk 40 does not necessarily have to be arranged in the transition area 14. However, one or more disks 40 arranged in the transition area 14 can support the expansion of a shape memory workpiece 100 with a targeted expansion angle 60, whereby a particularly reliable method for shaping a shape memory workpiece 100 can be provided. Adjoining the transition area 14 and following in the opposite direction of the axial direction a, a discrete diameter area 16 can extend, for example, within which at least two disks 40 in particular having the same diameter can be arranged.

The opposite direction of the axial direction a referred to above is a direction that is substantially parallel and opposite to the axial direction a.

The disks 40 in the discrete diameter area 16 can in particular have a larger diameter with respect to the disks 40 in the receiving area 12. The disks 40 arranged in the discrete diameter area 16 can form a second discrete diameter with the expansion wires 50 arranged or tensioned on them, in order to expand a shape memory workpiece 100 to a corresponding diameter.

The ratio of the average diameter of the transition area 14 to the diameter of the disks in the receiving area 12 can preferably be in the range from about 1.5 to about 1.9, particularly preferably about 1.85. Furthermore, the ratio of the diameter of the disks 40 in the discrete diameter area 16 to the mean diameter of the transition area 14 can preferably be in the range from about 1.5 to about 1.9, particularly preferably about 1.85.

In alternative embodiments, the ratio of the diameter of the disks in the discrete diameter area 16 to the diameter of the disks in the receiving area 12 can preferably be in the range from about 1.5 to about 1.9, more preferably about 1.85.

An actuator 32 is only outlined in FIG. 2 and is configured in particular to displace or move the disks 40 in and opposite to the substantially axial direction a or parallel to the traversing tube 30. To this end, on the one hand, the disks 40 can be arranged movably or traversably on the traversing tube 30 and can be moved directly by the actuator 32.

On the other hand, the disks 40 can be fixed or fixedly arranged on the traversing tube 30 and can be configured to be moved or displaced indirectly by the actuator 32, for example via the traversing tube 30. The movement or displacement by the actuator 32 is preferably configured as a movement or displacement substantially along the axial direction a or parallel to the traversing tube 30.

In further exemplary embodiments, a disk 40 can be provided with a shape in order to bring about a specific reshaping when the shape memory workpiece 100 passes, such as the formation of a hook on the shape memory workpiece 100.

Also indicated in FIG. 2 is a point that is marked as a magnification V. This point of the magnification V represents an exemplary portion of a disk 40 in more detail. The magnification V will be further explained in FIG. 3.

FIG. 3 shows a sketch of a portion of a disk 40 of an exemplary shaping tool 10. The sketch in FIG. 3 is reproduced as a partial sectional view of a disk 40, with several expansion wires or elements 50 also being shown in section in addition to the disk 40 for illustration purposes are. Also shown in FIG. 3 are the axial direction a pointing out of the drawing sheet, the radial direction r pointing radially outward starting from the axial direction a, and the circumferential direction u running in the circumferential direction u of the disk 40, which is circular at least in parts.

As shown in FIG. 3, each of the expanding wires or elements 50 can be arranged in at least one sliding groove 42, the sliding groove 42 preferably being arranged at a position along an outer circumference or along an outer circumferential side of the disk 40. The sliding groove 42 or the sliding grooves 42 can in particular have an opening that is open outward in the radial direction r, so that an expansion wire or element 50 arranged in the sliding groove 42 protrudes in the radial direction r with respect to the disk 40, in particular with respect to an outer circumference of the disk 40 or an outer circumferential side of the disk 40.

The protrusion of the expansion wire or element 50 with respect to the disk 40 can be made clear in particular by means of the overhang 46. In the sectional view according to FIG. 3, the overhang 46 describes a distance between the outermost point, in the radial direction r, of the expansion wire or element 50 with respect to an outermost point, in the radial direction r, of the disk 40, which adjoins the sliding groove 42. In exemplary embodiments, the overhang 46 can be about 5% to about 60%, preferably about 8% to about 40%, particularly preferably about 10% to about 20% with respect to the diameter or cross-section of the expansion wire or element 50. Advantageously, a sliding groove 42 designed such that it forms the overhang 46 with the expansion wire or element 50 arranged therein, which is less than 50% with respect to the diameter of the expansion wire 50, allows the expansion wire or element 50 to be radially secured or radially fixed in the sliding groove 42.

In exemplary embodiments, the sliding groove 42 of the disk 40 can be designed such that an expansion wire or element 50 arranged therein at least in parts can be guided in a sliding manner. In other words, the sliding groove 42 of the disk 40 can be formed such that the disk 40 can be displaced relative to the expansion wire 50. For this purpose, in exemplary embodiments, a sliding groove 42 can be formed in the axial direction a, in particular as a bore, which has a diameter or cross section that corresponds at least to the diameter or cross section of the expansion wire or element 50 arranged therein at least in parts. This advantageously enables a shape memory workpiece 100 to be arranged on a shaping tool 10 such that the shape memory workpiece 100 is held by the expansion wires or elements 50 and at the same time can be passed through by the disks 40, for example in the axial direction a or in a direction parallel to the traversing tube 30.

In preferred embodiments, the number of sliding grooves 42 corresponds at least to the number of expansion wires or elements 50 to be arranged on the disks 40, so that each expansion wire or element 50 can be arranged in a separate sliding groove 42.

In further preferred embodiments, the sliding grooves 42 are arranged substantially equidistantly on the outer circumference of the disk 40 or on an outer circumferential side of the disk 40, so that the shaping tool 10 enables a particularly uniform expansion of, in particular, round or substantially round workpieces. In alternative embodiments, the arrangement of the sliding grooves 42 can also deviate from an equidistant arrangement on the disk 40. Furthermore, in alternative embodiments, the sliding grooves 42 can be arranged on an inside of a disk, i.e., not on the outer circumference of the disk 40.

As further shown in FIG. 3, the disk 40 may have a concave portion 44 or a radially inwardly directed recess along its outer circumference or along its outer circumferential side and between two adjacent sliding grooves 42.

The concave portion 44 or the radially inwardly directed recess between two adjacent sliding grooves 42 advantageously ensures that in the case of a relative movement of the disks 40 in the axial direction a relative to the expansion wires 50, contacting of a shape memory workpiece 100 arranged on the expansion wires 50 and of the disks 40 can be prevented. This further allows the axial extension of the shape memory workpiece 100 to be advantageously maintained during expansion, in addition to preventing contamination from contact between the shape memory workpiece 100 and the disks 40 at high temperatures.

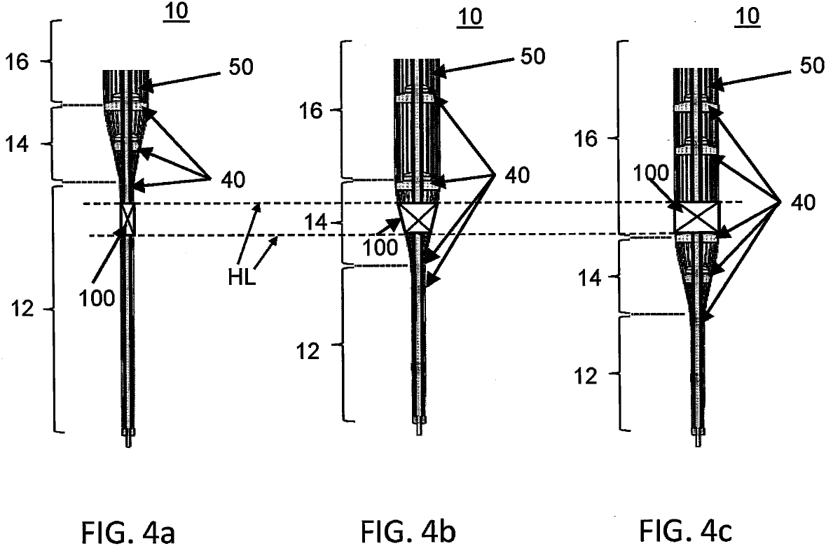

FIGS. 4a, 4b and 4c show a portion of an exemplary shaping tool 10 in a first state (FIG. 4a), a second state (FIG. 4b), and in a third state (FIG. 4c). Basically, FIGS. 4a, 4b and 4c show which positions the disks 40 and the plurality of expansion wires or elements 50 can assume in different steps of a method for shaping a shape memory workpiece 100. A schematically outlined shape memory workpiece 100 is shown arranged on the shaping tool 10 by way of example.

As indicated in FIGS. 4a, 4b and 4c, the expansion wires or elements 50 can be fixed in particular on a lower fixing portion of the shaping tool 10. In particular, the distal ends of the expansion wires or elements 50 can preferably be fixed to the lower fixing portion of the shaping tool 10. In exemplary embodiments, but not shown in the figures, the expanding wires or elements 50 can extend in particular between an upper fixing portion and a lower fixing portion of the shaping tool 10 and can preferably be stretched between the upper fixing portion and the lower fixing portion. The upper and lower fixing portions can be formed on the guide element 20, for example, but are not limited thereto.

In further exemplary embodiments, which are not shown in the figures, however, the expansion wires or elements 50 can in particular be guided back at a lower end of the guide element 20, in particular toward an upper fixing portion of the shaping tool 10.

In preferred embodiments, not shown in the figures though, a first end and a second end of the expansion wires or elements 50, in particular all or both ends of the expansion wires or elements 50, can be attached to an upper fixing portion or to an upper end of the shaping tool 10, with the expansion wires or elements 50 preferably being guided back at a lower end of the guide element 20. This advantageously makes it possible that no fixing, in particular no mechanical fixing, of the expansion wires or elements 50 is required at the lower end of the shaping tool 10. A further advantageous result of this is that the shaping tool 10 is also suitable for attaching particularly small shape memory workpieces 100, since the attachment of the shape memory workpiece 100, in particular to a lower portion of the shaping tool 10, in particular to the receiving area 12, is not affected by a fixation of the expansion wires or elements 50. Even more advantageously, this improves the durability and maintenance of the shaping tool 10 since the fixation of the expansion wires or elements 50 is not necessarily subject to direct heating by a heating device such as a salt bath.

FIG. 4a shows a first state of the shaping tool 10, which describes an example of a state of the shaping tool 10 when it is suitable for a shape memory workpiece 100 to be placed thereon. To this end, the shaping tool 10 can have a pronounced receiving area 12, which extends e.g., on a lower portion of the shaping tool 10 in the axial direction a, with the expansion wires or elements 50 forming a diameter that is suitable for arranging a shape memory workpiece 100 having a first or initial diameter thereon. In addition to the receiving area 12, the shaping tool 10 can further have a transition area 14 and a discrete diameter area 16, which adjoin or extend from the receiving area 12 in this order opposite to the axial direction a.

FIG. 4b shows a second state of the shaping tool 10 which, by way of example, describes a state of the shaping tool 10 when the shape memory workpiece 100 has been expanded to a second diameter. The expansion wires or elements 50 form a diameter in the transition area 14 which the shape memory workpiece 100 or portions of the shape memory workpiece 100 has or have after a first expansion, for example.

FIG. 4c shows a third state of the shaping tool 10, which, by way of example, describes a state of the shaping tool 10 when the shape memory workpiece 100 has been expanded to a third diameter. The expansion wires or elements 50 in the discrete diameter area 16 preferably form a diameter which the shape memory workpiece 100 has after a second expansion.

For the expansion itself, and as illustrated in a comparison between FIGS. 4a, 4b and 4c, at least the disks 40, which form the transition area 14 and the discrete diameter area 16, are preferably displaced or moved downward in the axial direction a, so that they preferably pass the shape memory workpiece 100 or pass the initial receiving area on which the shape memory workpiece 100 was originally arranged. The shape memory workpiece 100 can advantageously be expanded uniformly by passing of the disks 40. As is further illustrated by a comparison of FIGS. 4a, 4b and 4c, the shape memory workpiece 100 can be arranged or held at substantially the same position on the shaping tool 10 even after a step of expansion, and its axial extension can be maintained as highlighted in FIGS. 4a, 4b, and 4c by means of the dashed auxiliary lines HL.

FIGS. 4a, 4b and 4c are only schematic representations which, for the sake of clarity, do not show the entire shaping tool 10. The shape memory workpiece 100 arranged on the shaping tool 10 is also shown only schematically and not true to scale, in particular with regard to the axial extension.

In further exemplary embodiments of the shaping tool 10 that are not shown, it can have one or more further transition areas 14 and one or more further discrete diameter areas 16, which extend from the discrete diameter area 16 shown opposite to the axial direction a.

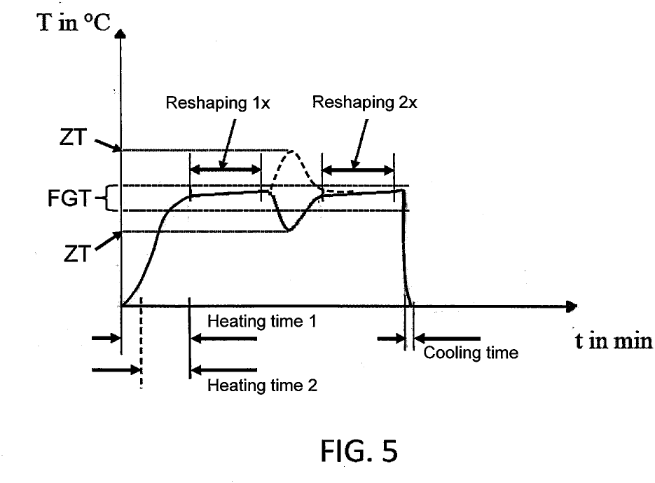

FIG. 5 shows a sketched temperature-time profile according to an example of the present method. The temperature-time profile shown in FIG. 5 for a method for shaping a shape memory workpiece 100 is to be considered a merely qualitative sketch and overview of how a method for shaping a shape memory workpiece 100 can take place.

As shown in FIG. 5, the shape memory workpiece 100 is preferably first warmed or heated to a shaping temperature FGT or to a temperature that is in the range of the shaping temperature FGT for the shape memory workpiece 100. While the shape memory workpiece 100 is in the temperature range of the shaping temperature FGT, it can preferably be deformed or reshaped, in other words expanded (shown in FIG. 5 with “reshaping 1x”). The degree of reshaping can be defined as desired, but is preferably in a range below about 1.9, particularly preferably about 1.85.

As further illustrated in FIG. 5, the shape memory workpiece 100 is cooled or further heated following a first reshaping or expansion, preferably to a temperature below or above the shaping temperature FGT. This temperature below or above the shaping temperature FGT can be referred to as the intermediate temperature ZT and can be below 500° C., for example, in particular in the range from about 250° C. to about 500° C., or above 525° C., particularly in the range of about 525° C. to about 600° C.

Subsequently, the shape memory workpiece 100 is brought back to the shaping temperature FGT, i.e., heated or cooled, in order to then be deformed or reshaped again. If the shape memory workpiece 100 has the desired target diameter after the second reshaping (shown in FIG. 5 with “reshaping 2x”), which is to be impressed on the shape memory workpiece 100 as a shape memory, it can then be cooled or quenched (in FIG. 5 illustrated by the short “cooling time”), e.g., cooled or quenched to about room temperature.

From the combination of multiple deformation of the shape memory workpiece 100 at the shaping temperature FGT, together with the intermediate change to a temperature below or above the shaping temperature FGT, and the final cooling as soon as the shape memory workpiece 100 has been expanded or reshaped to its target shape or diameter, predetermined shape memory properties or superelastic properties, comprising a desired diameter assumed at a predetermined ambient temperature and a predetermined load state, are impressed on the shape memory workpiece 100.

Furthermore, two heating durations for heating the shape memory workpiece 100 to the shaping temperature FGT are shown in FIG. 5 by way of example. The “heating time 1” represents the period of time that is required when the shaping tool 10 is not preheated, i.e., the method for shaping by means of the shaping tool 10 is started at about room temperature. In contrast, the “heating time 2” represents a shorter period of time if the shaping tool 10 has already run a shaping cycle with a shape memory workpiece 100. The comparatively shortened “heating time 2” is made possible in particular by the fact that the shape memory workpiece 100 is ejected from the shaping tool 10 for final cooling, as a result of which the shaping tool 10 itself does not undergo any significant cooling, in contrast to the ejected shape memory workpiece 100.

List of Reference Numerals

| | |
|---|---|
| 10 | shaping tool |
| 12 | receiving area |
| 14 | transition area |
| 16 | discrete diameter area |
| 20 | guide element |
| 30 | traversing tube |
| 32 | actuator |
| 40 | disk |
| 42 | sliding groove |
| 44 | concave portion |
| 46 | overhang |
| 50 | expansion wire |
| 60 | expansion angle |
| 100 | shape memory workpiece |
| S10–S90 | steps for a method for shaping a shape memory workpiece |
| a | axial direction |
| r | radial direction |
| u | circumferential direction |
| FGT | shaping temperature |
| HL | auxiliary line |
| V | magnification |
| ZT | intermediate temperature |

1. A method for shaping a shape memory workpiece, comprising:
providing a shape memory workpiece having a first diameter and a predetermined shaping temperature;
arranging the shape memory workpiece on a shaping tool;
heating the shape memory workpiece to the shaping temperature;
first expansion of the shape memory workpiece to a second diameter that is larger than the first diameter;
first changing of the temperature of the shape memory workpiece to an intermediate temperature below or above the shaping temperature;
bringing the shape memory workpiece to the shaping temperature again;
second expansion of the shape memory workpiece to a third diameter that is larger than the second diameter;
ejecting the shape memory workpiece from the shaping tool; and
final cooling of the shape memory workpiece to a cooling temperature below the intermediate temperature.

2. The method for shaping a shape memory workpiece according to claim 1, wherein the first changing of the temperature of the shape memory workpiece to an intermediate temperature comprises a first cooling of the shape memory workpiece to the intermediate temperature below the shaping temperature, or a first further heating of the shape memory workpiece to the intermediate temperature above the shaping temperature.

3. The method for shaping a shape memory workpiece according to claim 2, wherein the first changing of the temperature of the shape memory workpiece comprises changing the temperature of the shape memory workpiece by at least about 25° C.

4. The method for shaping a shape memory workpiece according to claim 2, wherein the first changing of the temperature of the shape memory workpiece comprises changing the temperature of the shape memory workpiece by at least about 40° C.

5. The method for shaping a shape memory workpiece according to claim 1, wherein the second diameter is about 1.5 times to about 1.9 times the first diameter; and/or the third diameter is about 1.5 times to about 1.9 times the second diameter.

6. The method for shaping a shape memory workpiece according to claim 1, wherein the second diameter is about 1.85 times the first diameter.

7. The method for shaping a shape memory workpiece according to claim 1, wherein the third diameter is about 1.85 times the second diameter.

8. The method for shaping a shape memory workpiece according to claim 1, wherein the first expansion and the second expansion comprise an expansion of the shape memory workpiece in the radial direction, in particular while maintaining the axial extension of the shape memory workpiece.

9. The method for shaping a shape memory workpiece according to claim 1, wherein the shape memory workpiece is held in a frictional manner during at least one of the first and second expansions.

10. The method for shaping a shape memory workpiece according to claim 1, wherein the shape memory workpiece:
has a nickel-titanium alloy; and/or
has a round shape; and/or
has a stent pattern.

11. A shaping tool for shaping a shape memory workpiece, having:
a guide element, and
a traversing tube, which is movably arranged on the guide element, wherein
disks are arranged on the traversing tube at predetermined axial distances,
expansion wires or expansion elements are stretched between the disks, and wherein
the expansion wires or expansion elements stretched between the disks form diameters in order to arrange a shape memory workpiece circumferentially thereon.

12. The shaping tool for shaping a shape memory workpiece according to claim 11, wherein the shaping tool has a receiving area for arranging thereon a shape memory workpiece having a first diameter, and wherein
- the traversing tube is configured to be moved relative to the receiving area by means of an actuator, so that the shape memory workpiece can be passed by the disks in the receiving area.

13. The shaping tool for shaping a shape memory workpiece according to claim 11, wherein the disks are shaped such that the shaping tool is configured to expand the shape memory workpiece by moving the traversing tube along the guide element.

14. The shaping tool for shaping a shape memory workpiece according to claim 11, wherein the disks have sliding grooves along their circumference,
wherein an expansion wire is slidably guided by the respective disk along each sliding groove, and
wherein each expansion wire is preferably arranged in the respective sliding groove such that it projects radially outward with respect to the disk in the sliding groove of which the expansion wire is guided.

15. The shaping tool for shaping a shape memory workpiece according to claim 11, having:
a heating device, preferably a heatable salt bath; and/or
a cooling device, preferably a water bath.

16. The shaping tool for shaping a shape memory workpiece according to claim 15, wherein the heating device is a heatable salt bath.

17. The shaping tool for shaping a shape memory workpiece according to claim 15, wherein the cooling device is a water bath.
